(19) United States
(12) Patent Application Publication
(10) Pub. No.: US 2017/0344833 A1
(43) Pub. Date: Nov. 30, 2017

(54) METHOD AND SYSTEM FOR IDENTIFYING AN INDIVIDUAL WITH INCREASED BODY TEMPERATURE

(71) Applicant: Imafor AB, Norrkoping (SE)

(72) Inventor: Jorgen Ahlberg, Linkoping (SE)

(73) Assignee: Imafor AB, Norrkoping (SE)

(21) Appl. No.: 15/531,917

(22) PCT Filed: Dec. 3, 2015
(86) PCT No.: PCT/EP2015/078600

§ 371 Date: May 31, 2017

(30) Foreign Application Priority Data
Dec. 5, 2014 (SE) 1451484-8

(51) Int. Cl. G06K 9/00; A61B 5/01; G06K 9/20; G01J 5/00

(52) U.S. Cl. CPC G06K 9/00771; G06K 9/2036; A61B 5/015; G01J 5/0022; G06K 2009/00939; G06K 9/00281

(57) ABSTRACT

A method of identifying one or more individuals with increased body temperature in a group of individuals, the method comprising the steps of: providing a scene (50), comprising one or more individuals, acquiring a plurality of successive sets of images of the scene (60), a set comprising a visual image and a thermal image, wherein in a set of images a content of the thermal image corresponds to a content of the visual image, detecting a feature of at least one of the individuals in at least one of the visual images (70), tracking at least one of the individuals based on the detected feature in the respective visual images of at least two of the sets of images (80), selecting a subset of the sets of images (100), using the feature in determining a measured temperature associated with the individual based on at least one of the thermal images of the subset (110), if the measured temperature exceeds a threshold value, providing an alarm (120), wherein providing the alarm comprises indicating the individual(s) associated with the alarm (130).

### TECHNICAL FIELD

The present disclosure relates to a method and system of identifying one or more individuals with increased body temperature in a group of individuals.

### TECHNICAL BACKGROUND

Fever screening is of increasing interest in today's world of global travel and transportation of humans and animals, as outbreaks of infectious diseases (SARS, bird flu, swine flu, H₁N₁, Ebola virus etc.) may cross national and international boundaries in a matter of hours.

Fever screening is today often performed by photographing one individual at a time at a predetermined position with a thermal camera. From the thermal image it is then determined if the current individual has fever or not.

Fever screening is for example described in U.S. Pat. No. 7,548,776, which document discloses a method and system for performing fever triage in which a thermal image-detecting unit and a visual light image-detecting unit are used to detect a single testee at a same time-point and position so as to form thermal image data and visual light image data. The thermal image data is used to determine if there in at least a portion of the thermal image is an increased temperature within a predetermined temperature range. The visual light image is used to determine if the portion of the thermal image identified with increased temperature corresponds to the skin portions of the visible light image. If this is the case, a warning is raised to indicate that the testee has fever.

Such a system is suitable for example at an airport in which disembarking passengers are narrowed down into a single file along the aerobridge/screening point. As they walk through one by one, each passenger is screened for fever and potentially feverish passengers are filtered out for further medical tests. The rest walk on and continue as normal.

Such a system is, however, not suitable for mass screening as only one person at a time is analyzed for increased temperature and passenger flow is not maintained in high volume pedestrian transit areas.

### SUMMARY OF THE INVENTION

It is an object of the present disclosure to provide methods and systems for fever screening, which are suitable for mass screening without disturbing the passenger flow. It is also an object to provide methods and systems for fever screening which are reliable such that those individuals with fever are identified as having fever and those individuals without fever are not falsely identified as having fever.

The invention is defined by the appended independent claims. Embodiments are set forth in the dependent claims, in the attached drawings and in the following description.

According to a first aspect there is provided a method of identifying one or more individuals with increased body temperature in a group of individuals, the method comprising the steps of: providing a scene, comprising a plurality of individuals, acquiring a plurality of successive sets of images of the scene, a set comprising a visual image and a thermal image, wherein in a set of images a content of the thermal image corresponds to a content of the visual image, detecting a feature of at least one of the individuals in at least one of the visual images, tracking the at least one of the individuals based on the detected feature in the respective visual images of at least two of the sets of images, and selecting a subset of the sets of images for the at least one individual being tracked. The feature is used in determining a measured temperature associated with the individual based on at least one of the thermal images of the subset, and if the measured temperature exceeds a threshold value, providing an alarm, wherein providing the alarm comprises indicating the individual(s) associated with the alarm.

With individual is meant an individual selected from vertebrates, such as poultry, or mammals, such as human beings, cows, pigs etc.

With a plurality of individuals is here meant, at least two individuals, at least three individuals etc. up to 500 individuals.

The term “corresponds” may be understood as depicting substantially the same scene. Hence, minor deviations in viewing angle and/or time of image acquisition may be tolerated, though ideally, the images are taken simultaneously and along a common optical axis or along parallel axes with a known calibration.

With minor deviation in viewing angle is here meant a deviation of maximum around 10 degrees. As long as there is a substantial overlap of the images, a deviation in viewing angle may be compensated for.

With minor deviation in time of image acquisition is here meant fractions of a second, e.g. 0 to +/−0.5 seconds, 0 to +/−0.1 seconds or 0 to +/−0.05 seconds.

A visual image is understood as a digital image displaying actual, or close to actual, colors or shades of colors corresponding to their real colors.

A thermal image is understood as a digital image showing differences in temperature, with each color or grey scale level corresponding to a specific temperature.

The term “successive” indicates that the sets are acquired at different points in time. The sets may, but need not, be acquired as immediately successive sets. A subset may include one or more of the sets. Preferably each set of images comprises a visual image and a thermal image.

Indicating the individual associated with the alarm by providing an indication based on the visual image may be done by cutting out a part of the visual image, optionally enlarged, and display the visual image for identification purposes. In the alternative, a larger portion of the visual image may be displayed, with the individual being indicated by means of a marker, such as a box, a ring, an arrow, a highlight etc.

With this system it is possible to identify one or more individuals with increased body temperature from a group of individuals, measure the body surface temperature of by-passing individuals or of a plurality of simultaneously by-passing individuals, alerting if an increased body surface temperature is detected in an individual and indicating the individual who caused the alert.

The method may further comprise a step of determining an orientation of a feature associated with the individual that is being tracked, and selecting the subset based on the orientation.

The feature may especially for a human being be an individual's face, though other body parts presenting sufficiently unique features which can be distinguished at sufficient distance may be used instead or in addition to an individual's face.

The method may further comprise a step of determining a size of a feature associated with the individual that is being tracked, and selecting the subset based on the size.

The method may further comprise a step of determining a distance to a feature associated with the individual that is being tracked, and selecting the subset based on the distance.

Detecting a feature may comprise detecting a face of the individual, preferably detecting at least one eye of the individual.

Detecting a feature may further comprise detecting at least one corner of the individual's eye, preferably a corner closest to the individual's nose. The step of indicating the individual associated with the alarm may comprise providing an indication directly towards the individual.

A direct indication may be a beam of light, or any other type of marking mechanism, such as paint (paintball gun).

As an alternative, or in addition, to providing an alarm and indicating the individual(s) associated with such alarm, it is possible to save data on the number of individuals for whom the measured temperature exceeds a threshold value and on the number of individuals for whom the measured temperature does not exceed the threshold value. An alarm may be triggered if the ratio of high-temperature individuals to normal temperature individuals increases above a certain level, which could indicate an epidemic outbreak.

Such data could be associated with the individuals, e.g. through the association with the visual image and any other identification data that may be available, as the case may be. In the alternative, such data could be on an anonymous basis

According to a second aspect there is provided a method of identifying one or more individuals with increased body temperature in a group of individuals, the method comprising the steps of: providing a scene, comprising a plurality of individuals, acquiring a plurality of successive sets of images of the scene, a set comprising a visual image and a thermal image, wherein in a set of images a content of the thermal image corresponds to a content of the visual image, detecting a feature of at least one of the individuals in at least one of the visual images and thermal images, tracking the at least one of the individuals based on the detected feature in the respective visual images and/or the thermal images of at least two of the sets of images, and selecting a subset of the sets of images for the at least one individual being tracked. The feature is used in determining a measured temperature associated with the individual based on at least one of thermal images of the subset, and if the measured temperature exceeds a threshold value, providing an alarm, wherein providing the alarm comprises indicating the individual(s) associated with the alarm based on the visual image(s).

Tracking may in the method of this aspect be based on the thermal image(s), the visual image(s) or both the thermal and visual image(s).

Detection of a feature of an individual(s) in the method of this aspect is made in the visual image(s), in the thermal image(s) or in both the thermal and visual image(s).

That the alarm is based on the visual image facilitates identification of the individual causing the alarm if the individual for example is a human being in a group of other human beings. In addition, visual imaging devices in general result in images with a higher resolution than images from thermal imaging devices.

According to a third aspect of the invention there is provided a method of identifying one or more individuals in a group of individuals, the method comprising the steps of: providing a scene comprising one or more individuals, acquiring a plurality of successive sets of images of the scene, a set comprising a visual image and a thermal image, wherein in a set of images a content of the thermal image corresponds to a content of the visual image, selecting a subset of the sets of images and performing a feature detection based on at least one of the images of the subset, and if the feature detection detects presence of at least one individual but fails to detect a predetermined feature associated with that individual, providing an alarm, wherein providing the alarm comprises indicating the individual(s) associated with the alarm.

This method may further comprise using the detected predetermined feature in determining a measured temperature associated with the individual based on at least one of the thermal images of the subset (110), and if the measured temperature exceeds a threshold value, providing an alarm (120), wherein providing the alarm comprises indicating the individual(s) associated with the alarm (130).

The method may further comprise tracking at least one of the individuals in at least two of the sets of images.

According to a fourth aspect of the invention there is provided a system for identifying an individual with increased body temperature in a group of individuals, the system comprising: a thermal imaging device arranged to provide thermal images of a scene comprising a plurality of individuals, a visual imaging device arranged to provide visual images of said scene, wherein the thermal and visual imaging devices are arranged in such a way that a content of a thermal image corresponds to a content of the visual image in a set of images of the scene, and a processing device arranged to detect a feature of at least one of the individuals in at least one of the visual images, track at least one of the individuals based on the detected feature in the respective visual images of at least two of the sets of images, select a subset of the sets of images for the at least one individual being tracked and use the feature in determining a measured temperature associated with the individual based on at least one of the thermal images, and provide an alarm if the measured temperature exceeds a threshold value and to indicate the individual(s) associated with the alarm.

According to a fifth aspect of the invention there is provided a system for identifying an individual with increased body temperature in a group of individuals, the system comprising: a thermal imaging device arranged to provide thermal images of a scene comprising a plurality of individuals, a visual imaging device arranged to provide visual images of said scene, wherein the thermal and visual imaging devices are arranged in such a way that a content of a thermal image corresponds to a content of the visual image in a set of images of the scene and a processing device arranged to detect a feature of at least one of the individuals in at least one of the visual images and thermal images, track at least one of the individuals based on the detected feature in the visual images and/or thermal imaged of at least two of the sets of images, select a subset of the sets of images for the at least one individual being tracked and use the feature in determining a measured temperature associated with the individual based on at least one of the thermal images, and provide an alarm if the measured temperature exceeds a threshold value and to indicate the individual(s) associated with the alarm based on the visual image(s).

### BRIEF DESCRIPTION OF THE DRAWINGS

FIG. 1 shows schematically a method of identifying and indicating one or more individuals with increased body temperature in a group of individuals.

FIG. 2a shows a group of individuals and a system for identifying one or more individuals with increased body temperature in a group of individuals.

FIG. 2b shows an enlarged image of the group of individuals in FIG. 2a, in which image an individual detected with increased body temperature is indicated.

FIGS. 3a and 3b shows two alternative ways of arranging the thermal and visual imaging devices such that a content of the thermal image corresponds to a content of the visual image.

FIG. 4a shows a thermal image of a group of people and FIG. 4b its corresponding visual image.

### DETAILED DESCRIPTION

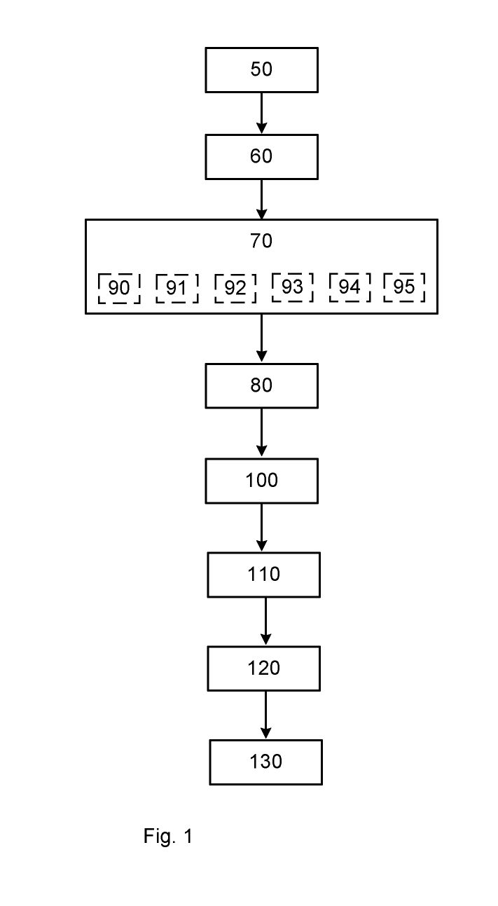

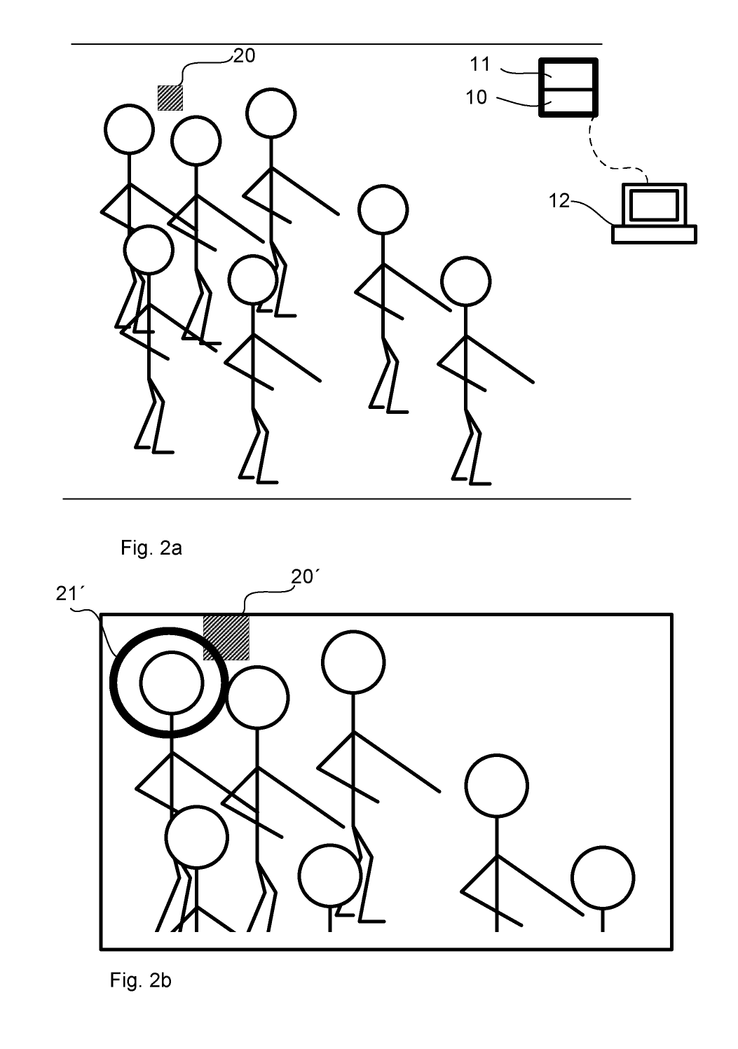

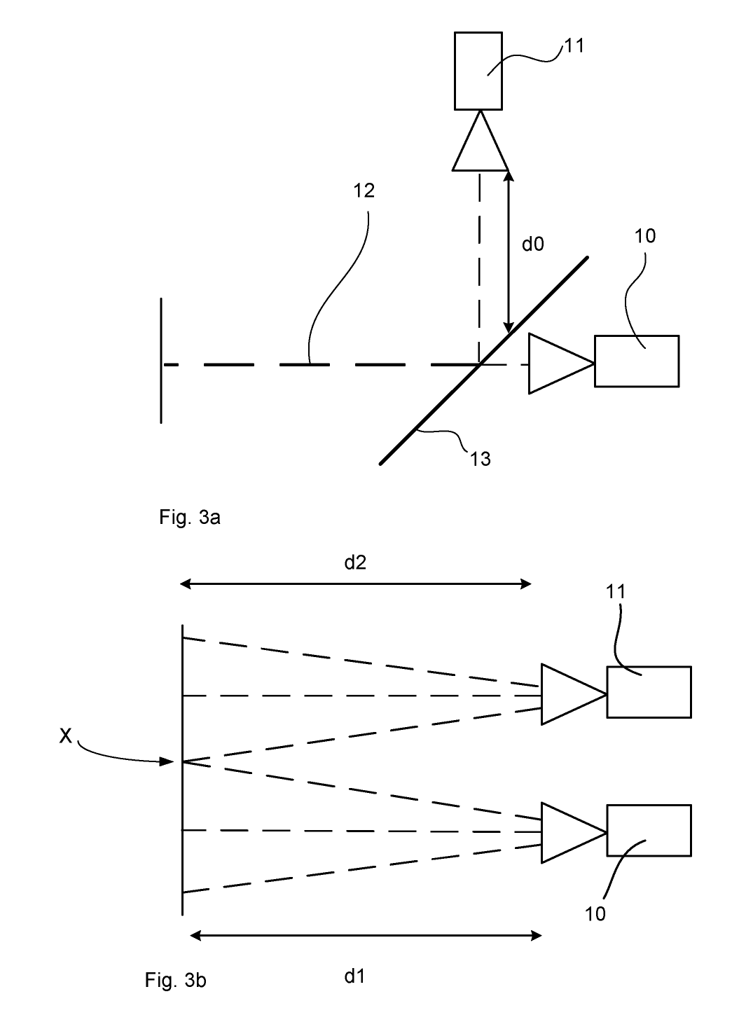

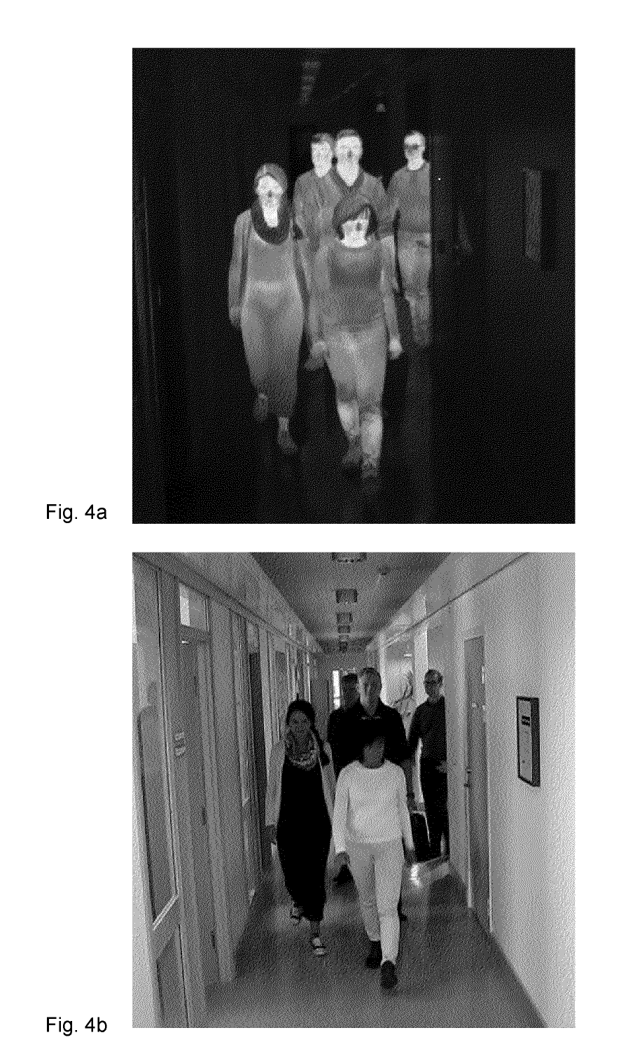

A method of identifying one or more individuals with increased body temperature in a group of individuals is illustrated in FIG. 1. With individual is meant vertebrates, such as poultry, or mammals, such as human beings, cows, pigs etc. The method comprises the steps of providing a scene, comprising one or more individuals 50, see FIG. 2a. A plurality of successive sets of images is acquired of the scene 60. The successive images are acquired at different points in time and may be acquired as immediately successive sets. A set, or each set of images, comprises a visual image (see FIG. 4b), i.e. an image displaying actual colors, and a thermal image (see FIG. 4a), i.e. an image showing differences in temperature, with each color corresponding to a specific temperature, acquired by means of a visual imaging device 10 and a thermal imaging device, respectively. A thermal imaging device 11 (FIG. 2a, 3a, 3b) is an infrared (IR) camera. A visual imaging device is for example a normal camera 10 (FIG. 2a, FIGS. 3a and 3b).

In a set of images a content of the thermal image corresponds to a content of the visual image, i.e. the images depict substantially the same motive. Hence, minor deviations in viewing angle and/or time of image acquisition may be tolerated, though ideally, the images are taken simultaneously and along a common optical axis or along parallel axes with a known calibration.

Adjusting the thermal imaging device 10 and the visual imaging device 11 along a common optical axis 12, see FIG. 3a, is for example accomplished by placing a disc 13, which is of a material, such as glass, which is translucent for the visual imaging device 10 and reflecting for the thermal imaging device 11, in front of the visual imaging device 10 and with an angle of about 45° against the optical axis of the visual imaging device 10. The thermal imaging device 11 is arranged at a distance d0 from the visual imaging device 10 and directed in such a way that its optical axis is deflected by the disc 13 and coincides with the optical axis of the visual imaging device.

Thereby, the visual and thermal imaging devices 10, 11 obtain a common optical axis 13. This procedure is per se known.

Calibrating the thermal imaging device 11 and the visual imaging device 10 comprises arranging the thermal imaging device 11 and the visual imaging device 10 so that for any point X on a plane at a certain distance d1 from the visual imaging device 10 and at a certain distance d2 from the thermal imaging device 11, respectively, see FIG. 3b, the corresponding points in the visual and thermal images are known. This procedure is per se known. The distances d1 and d2 describe the optimal focus distance.

In the next step in the method (FIG. 1), a feature is detected of at least one of the individuals in at least one of the visual images and/or thermal images 70.

The detected feature may be a face 93 of an individual. Methods for detecting a face or faces in thermal images and/or visual images are per se known. One such method is for example discussed in N. Marku\check{s}, M. Frljak, I. S. Pandzic, J. Ahlberg, and R. Forchheimer, “A method for object detection based on pixel intensity comparisons”, arXiv:1305.4537 [cs.CV], May 2013. It is to be clear that other kinds of known face detection methods could be used in the present method.

If an individual is wearing a veil, or is wholly or partially covering his/her face in any other way or is turning the face in a direction away from the thermal and visual imaging devices, face detection is not possible. In such cases detection of individuals as such may be performed. Methods for detecting an individual in thermal images and/or visual images are per se known. One such method is for example discussed in P. Dollar, C. Wojek, B. Schiele and P. Perona, “Pedestrian Detection: An Evaluation of the State of the Art”, IEEE Transactions on Pattern Analysis and Machine Intelligence, Volume 34, Issue 4, pp. 743-761, April 2012. It is to be understood that other kinds of methods for detecting individuals could be used.

It is to be clear that other kinds of known methods for detection of individuals could be used in the present method.

If no face is detectable for the detected individual a warning may be given that for this individual face detection is not possible and that body temperature has not been measured. Such a warning could for example comprise visualization based on the visual image and may comprise cutting out a part of the visual image, optionally enlarged, and displaying the visual image for identification purposes. A user may then look at the visual image and pick out individuals corresponding to those indicated for e.g. further measurement. Alternatively, a direct indication of the individual not measured by using of a beam of light, or any other type of marking mechanism, such as paint (paintball gun) may be used. Such indication could be useful if the method is used for example in detecting increased temperature in poultry, cattle or pigs.

Instead of, or in combination with face detection 93, eye detection 94 maybe used to detect at least one eye of the individual. In this method detection of a corner of the individual's eye 95, preferably a corner closest to the individual's nose, may be used in an alternative embodiment.

Methods of eye detection in the thermal image and/or in the visual image are known per se. One such method is for example discussed in N. Marku\check{s}, M. Frljak, I. S. Pandzic, J. Ahlberg, and R. Forchheimer, “Eye pupil localization with an ensemble randomized trees,” Pattern Recognition, Vol. 47, Issue 2, pp. 578-587, February 2014. DOI: 10.1016/j.patcog.2013.08.008. It is to be understood that other kinds of eye detection methods could be used.

As human skin temperature varies with environment and physiological factors, measuring temperature for example at the forehead does not always accurately reflect internal core temperature, as raised skin temperature can occur in the absence of fever and vice versa. When measuring in the eye area this problem may be decreased especially when measuring temperature in a corner of the eye a good indication of internal core temperature is provided. Further, according to the ISO standard for thermal camera use in fever screening, ISO 80601-2-56:2009, measurement of temperature should be made in the inner corner of the eye.

In some embodiments of the method it is, however, possible to measure for example skin temperature on the forehead of an individual. This could for example be of interest if the individual to be measured wears glasses, as presence of glasses hinder thermal images of the eye area of the individual.

Alternatively, when an individual wearing glasses, and thereby hindering thermal images of the eye area of the individual, is present in the scene, a warning may be given that for this individual the temperature has not been measured. Such a warning may be provided in the manners disclosed above.

In another step in the method, at least one of the individuals is tracked based on the detected feature in the respective visual images and/or thermal images of at least two of the sets of images 80.

By tracking 80 individuals, an already detected individual will not if identified with an increased body temperature give rise to an alarm twice, as new images of an individual are associated with earlier images of that individual.

The step of tracking individuals is a per se known method. One such method is for example discussed in S. Blackman and R. Popoli, Design and Analysis of Modern Tracking Systems, Artech House, Norwood Mass., 1999. It is to be understood that other kinds of known face tracking methods could be used in the method. Such methods include methods that do not separate feature detection and tracking of individuals, i.e. feature detection need not be performed prior to tracking.

Next in the method, a subset of the sets of images is selected 100. The subset may be selected based on one or more of the following:
a) Orientation of the feature associated with the individual (s) that is being tracked 90 and derived from the visual images and/or thermal images.
It is for example preferable that a detected face is turned towards the thermal and/or the visual imaging device 11, 10 during detection, i.e. an angle between the face and the optical axle is small enough. This angle may be estimated by adapting a face model to measured points or by adapting an appearance model to the image. This procedure is per se known.
b) Size of the feature associated with the individual (s) that is being tracked 91.
It is advantageous if the size of the detected face in a thermal image is big enough for allowing temperature measurement in a small enough area of the detected face, i.e. that a certain area or distance of the detected face corresponds to a certain amount of pixels.
c) A distance to the feature associated with the individual(s) that is being tracked (92).
The distance of the individual from the thermal imaging device and/or the visual imaging device should be within a certain distance interval, which distance interval may be estimated as it is correlated to the distance between for example the eyes of a detected face in a visual or thermal image.
   A subset may include one or more of the sets. Preferably each subset comprises a visual image and a thermal image.

Having selected a subset of images, the next step in the method (FIG. 1) is to use the feature in determining a measured temperature associated with the individual based on at least one of the thermal images of the subset 110.

This step may be performed by comparing the color, or actually a value corresponding to temperature, of the feature in the thermal image(s) with the color of at least one temperature reference point 20′ in the same thermal image(s), where the temperature reference point 20′ has a known temperature (see FIG. 2b).

A temperature reference point 20 may be arranged in a such a way that the thermal imaging device 11 when detecting a by-passing individual or a plurality of by-passing individuals also detects the temperature reference point 20 in the same thermal image, FIG. 2a, 2b.

The temperature reference point 20 may be a point with a known fixed temperature or a point with a known gradually changing temperature measured. The reference point 20 may be used to stabilize the temperature measurement.

When the selected subset of images comprises more than one thermal image, the temperature may be measured in the feature in all the thermal images. A final temperature value may be obtained by weighting and adding together the measured temperature values. Weights may be given on the following criteria:
a. high measured temperature value
b. detected feature turned towards the visual and/or thermal imaging device 10, 11
c. many pixels (measurement points) in the area of detection
d. images close to the optimal focus distance d2 from the thermal imaging device 11
e. if faces/eyes are detected by the visual imaging device

If the measured temperature exceeds a threshold value an alarm is provided 120. Providing the alarm 120 may comprise indicating the individual(s) associated with the alarm 130.

Alternatively, an alert is given if the measured temperature deviates from a statistical model. In such case a temperature reference is not necessary but might optionally be used to stabilize the measurements. In the statistical model an average body temperature of the individuals measured may be calculated and individuals with a body temperature exceeding this average temperature with a certain amount may be indicated. The statistical model may be built up in real time or may be a saved accumulated model. The statistical model may be correlated to the ambient temperature. It is to be understood that other kinds of statistical models than average value based models may be used in the method.

The step of indicating the individual identified with increased body temperature 130 may comprise visualizing the individual based on the visual image(s). Such visualization may comprise cutting out a part of the visual image, optionally enlarged, and displaying the visual image for identification purposes. A user may then look at the visual image and pick out individuals corresponding to those indicated for e.g. further measurement with more accurate equipment.

In the alternative, a larger portion of the visual image may be displayed, with the individual being indicated by means of a marker, such as a box, a ring 21′, an arrow, a highlight etc, see FIG. 2b.

Alternatively, or in combination with visualization based on the visual image, a direct indication of the individual detected with increased temperature may done be using of a beam of light, or any other type of marking mechanism, such as paint (paintball gun). Such indication could be useful if the method is used for example in detecting increased temperature in poultry, cattle or pigs.

The color and/or size and/or shape of the indicating light or the paint, could be adapted to for example give information about how much the measured increased temperature deviates from a normal temperature.

A system of identifying one or more individuals with increased body temperature in a group of individuals is illustrated in FIG. 2a. Such a system comprises a thermal imaging device 11, arranged to provide thermal images of a scene comprising one or more individuals, and a visual imaging device 10 arranged to provide visual images of said scene. The thermal and visual imaging devices are arranged, as described above, in such a way that a content of a thermal image corresponds to a content of the visual image in a set of images of the scene. The system further comprises a processing device 12, which is arranged to implement the method as described above.

The processing device 12 is connected to the thermal and imaging devices 11, 10 and may be provided with a memory, software, a display unit and an input and/or output unit.

With this method and system it is possible to identify one or more individuals with increased body temperature in a group of individuals and alert if an increased body temperature is measured in an individual and indicating the individual who caused the alert. The method and system are, hence, suitable for mass screening as they do not require that for example disembarking passengers are narrowed down into a single file along the aerobridge/screening point. The method/system does not disturb the passenger flow. Further, the method/system can be made reliable such that those individuals with fever are identified as having fever and those individuals without fever are not falsely identified as having fever.

1. Method of identifying one or more individuals with increased body temperature in a group of individuals, the method comprising the steps of:
providing a scene, comprising a plurality of individuals,
acquiring a plurality of successive sets of images of the scene, each set comprising a visual image and a thermal image, wherein, in each set of images a content of the thermal image corresponds to a content of the visual image,
detecting a feature in the form of a body part of at least one of the individuals in at least one of the visual images,
tracking the at least one of the individuals based on the detected feature in the respective visual images of at least two of the sets of images,
selecting a subset of the sets of images for the at least one individual being tracked,
using the feature in determining a measured temperature associated with the individual based on at least one of the thermal images of the subset,
if the measured temperature exceeds a threshold value, providing an alarm, wherein providing the alarm comprises indicating the individual(s) associated with the alarm.

2. Method of identifying one or more individuals with increased body temperature in a group of individuals, the method comprising the steps of:
providing a scene, comprising a plurality of individuals,
acquiring a plurality of successive sets of images of the scene, each set comprising a visual image and a thermal image, wherein, in each set of images a content of the thermal image corresponds to a content of the visual image,
detecting a feature in the form of a body part of at least one of the individuals in at least one of the visual images and thermal images,
tracking the at least one of the individuals based on the detected feature in the respective visual images and/or thermal images of at least two of the sets of images,
selecting a subset of the sets of images for the at least one individual being tracked,
using the feature in determining a measured temperature associated with the individual based on at least one of the thermal images of the subset,
if the measured temperature exceeds a threshold value, providing an alarm, wherein providing the alarm comprises indicating the individual(s) associated with the alarm based on the visual image(s).

3. The method as claimed in claim 1, further comprising:
determining an orientation of the feature associated with the individual (s) that is being tracked, and
selecting the subset based on the orientation.

4. The method as claimed in claim 1, further comprising:
determining a size of a feature associated with the individual(s) that is being tracked, and
selecting the subset based on the size.

5. The method as claimed in claim 1, further comprising:
determining a distance to a feature associated with the individual(s) that is being tracked (92), and
selecting the subset based on the distance.

6. The method as claimed in claim 1, wherein detecting a feature comprises detecting a face and an eye of the individual(s).

7. The method as claimed in claim 6, further comprising detecting at least one corner of the individual's eye.

8. The method as claimed in claim 1, wherein indicating the individual(s) associated with the alarm comprises providing an indication directly towards the individual(s).

9. The method as claimed in claim 1,6 wherein indicating the individual(s) associated with the alarm comprises providing an indication based on the visual image(s).

10. Method of identifying one or more individuals in a group of individuals, the method comprising the steps of:
providing a scene, comprising one or more individuals,
acquiring a plurality of successive sets of images of the scene, each set comprising a visual image and a thermal image, wherein, in each set of images a content of the thermal image corresponds to a content of the visual image,
selecting a subset of the sets of images,
performing a feature detection for detection of a body part based on at least one of the images of the subset, and
if the feature detection detects presence of at least one individual but fails to detect a predetermined feature of that individual, providing an alarm, wherein providing the alarm comprises indicating the individual(s) associated with the alarm.

11. The method as claimed in claim 10, further comprising:
using the detected predetermined feature in determining a measured temperature associated with the individual based on at least one of the thermal images of the subset,
if the measured temperature exceeds a threshold value, providing an alarm, wherein providing the alarm comprises indicating the individual(s) associated with the alarm.

12. The method as claimed in claim 10, further comprising tracking at least one of the individuals in at least two of the sets of images.

13. System for identifying one or more individuals with increased body temperature in a group of individuals, the system comprising:
a thermal imaging device configured to provide thermal images of a scene comprising a plurality of individuals,
a visual imaging device configured to provide visual images of said scene,
wherein the thermal and visual imaging devices are configured in such a way that a content of a thermal image corresponds to a content of the visual image in a set of images of the scene, and
a processing device configured to:
detect a feature in the form of a body part of at least one of the individuals in at least one of the visual images,
track at least one of the individuals based on the detected feature in the respective visual images of at least two of the sets of images,
select a subset of the sets of images for the at least one individual being tracked and use the feature in determining a measured temperature associated with the individual based on at least one of the thermal images, and
provide an alarm if the measured temperature exceeds a threshold value and to indicate the individual(s) associated with the alarm.

14. System for identifying one or more individuals with increased body temperature in a group of individuals, the system comprising:
a thermal imaging device configured to provide thermal images of a scene comprising a plurality of individuals,
a visual imaging device configured to provide visual images of said scene,
wherein the thermal and visual imaging devices are configured in such a way that a content of a thermal image corresponds to a content of the visual image in a set of images of the scene,
a processing device configured to
detect a feature in the form of a body part of at least one of the individuals in at least one of the visual images and thermal images,
track at least one of the individuals based on the detected feature in the visual images and/or thermal images of at least two of the sets of images,
select a subset of the sets of images for the at least one individual being tracked and use the feature in determining a measured temperature associated with the individual based on at least one of the thermal images, and
provide an alarm if the measured temperature exceeds a threshold value and to indicate the individual(s) associated with the alarm based on the visual image(s).
